(19) United States
(12) Patent Application Publication
(10) Pub. No.: US 2023/0240598 A1
(43) Pub. Date: Aug. 3, 2023

(54) NERVE CUFF WITH SIDE WING NEEDLES TO MONITOR EMG AND SIDE EFFECTS

(71) Applicant: Medtronic, Inc., Minneapolis, MN (US)

(72) Inventor: Kanthaiah Koka, Valencia, CA (US)

(21) Appl. No.: 18/072,489

(22) Filed: Nov. 30, 2022

Related U.S. Application Data
(60) Provisional application No. 63/306,415, filed on Feb. 3, 2022.

(51) Int. Cl. A61B 5/00; A61B 5/024

(52) U.S. Cl. CPC A61B 5/4836; A61B 5/024

(57) ABSTRACT

A system includes a first electrode, a second electrode, and a suture structure. The first electrode and the second electrode are both coupled to the suture structure. The system may deliver, via the first electrode, electrical stimulation signals to a nerve or nerve branch. The system may sense, via the second electrode, response signals based on delivering the electrical stimulation signals. The system may control parameters associated with delivering the electrical stimulation signals, based on sensing the response signals.

### CROSS REFERENCE TO RELATED APPLICATIONS

This application claims the benefit of U.S. Provisional Application No. 63/306,415, filed on Feb. 3, 2022, and entitled “Nerve Cuff with Side Wing Needles to Monitor EMG and Side Effects”, which application is incorporated herein by reference in its entirety.

### BACKGROUND

The present disclosure is generally directed to nerve stimulation, and relates more particularly to stimulation of a vagus nerve, a tibial nerve, or other peripheral nerves.

Some devices may support nerve stimulation for treating a medical condition. Improved techniques for delivering nerve stimulation and monitoring effects thereof are desired.

### BRIEF SUMMARY

Example aspects of the present disclosure include:

A device including: a cuff electrode; one or more needle electrodes; and a suture structure, where the cuff electrode and the one or more needle electrodes are both coupled to the suture structure.

Any of the aspects herein, wherein the suture structure includes a suture wing structure.

Any of the aspects herein, wherein the one or more needle electrodes include one or more needle hook electrodes; and a stability associated with a placement of the device is associated with the needle hook electrodes.

Any of the aspects herein, wherein the one or more needle electrodes include one or more active needle electrodes.

Any of the aspects herein, further including: electronic circuitry configured to: deliver, via the first electrode, one or more electrical stimulation signals to a nerve or a nerve branch; sense, via the one or more second electrodes, one or more signals based on delivering the one or more electrical stimulation signals; and control one or more parameters associated with delivering the one or more electrical stimulation signals, based on sensing the one or more signals.

Any of the aspects herein, wherein the one or more signals include one or more electromyographic (EMG) signals.

Any of the aspects herein, wherein the one or more signals include one or more electroneurographic (ENG) signals.

Any of the aspects herein, wherein the one or more signals include one or more electrocardiogram (EKG) signals.

Any of the aspects herein, wherein the one or more signals are representative of a blood glucose level.

A system including: a first electrode; one or more second electrodes; a suture structure, where the first electrode and the one or more second electrodes are both coupled to the suture structure; a processor; and a memory storing data thereon that, when processed by the processor, cause the processor to: deliver, via the first electrode, one or more electrical stimulation signals to a nerve; sense, via the one or more second electrodes, one or more signals based on delivering the one or more electrical stimulation signals; and control one or more parameters associated with delivering the one or more electrical stimulation signals, based on sensing the one or more signals.

Any of the aspects herein, wherein the suture structure includes a suture wing structure.

Any of the aspects herein, wherein the first electrode includes a cuff electrode.

Any of the aspects herein, wherein the one or more signals include one or more EMG signals.

Any of the aspects herein, wherein the one or more signals include one or more ENG signals.

Any of the aspects herein, wherein the one or more signals include one or more EKG signals.

Any of the aspects herein, wherein the one or more signals are representative of a blood glucose level.

Any of the aspects herein, wherein the one or more signals are representative of a feedback response of an anatomical element of a subject corresponding to the one or more electrical stimulation signals.

Any of the aspects herein, wherein the data, when processed by the processor, further causes the processor to: determine biometric information associated with a subject based on the one or more signals, where controlling the one or more parameters is based on determining the biometric information.

Any of the aspects herein, wherein the biometric information includes one or more biometric responses of the subject corresponding to the one or more electrical stimulation signals, the one or more biometric responses including at least one of: a neural response of the subject; a change to a heart rate of the subject; and one or more laryngopharyngeal symptoms of the subject.

Any of the aspects herein, wherein delivering the one or more electrical stimulation signals, sensing the one or more signals, and controlling the one or more parameters associated with delivering the one or more electrical stimulation signals is based on closed-loop control.

Any of the aspects herein, wherein the data, when processed by the processor, further causes the processor to: generate status information associated with a subject based on sensing the one or more signals, controlling the one or more parameters, or both; and provide at least a portion of the clinical data to the subject, a medical provider, or both.

Any of the aspects herein, wherein the one or more second electrodes include one or more needle electrodes.

Any of the aspects herein, wherein: the one or more second electrodes include one or more needle hook electrodes; and a stability associated with a placement of the first electrode and the suture structure is associated with the needle hook electrodes.

Any of the aspects herein, wherein the one or more second electrodes include one or more active electrodes.

Any of the aspects herein, wherein controlling the one or more parameters includes modifying at least one of: a duration associated with delivering the one or more electrical stimulation signals; a frequency of the one or more electrical stimulation signals; a pulse width of the one or more electrical stimulation signals; a duty cycle of the one or more electrical stimulation signals; and an amplitude of the one or more electrical stimulation signals.

A method including: delivering, via a first electrode, one or more electrical stimulation signals to a nerve; sensing, via one or more second electrodes, one or more signals based on delivering the one or more electrical stimulation signals, where the first electrode and the one or more second electrodes are both coupled to a suture structure; and controlling one or more parameters associated with delivering the one or more electrical stimulation signals, based on sensing the one or more signals.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The details of one or more aspects of the disclosure are set forth in the accompanying drawings and the description below. Other features, objects, and advantages of the techniques described in this disclosure will be apparent from the description and drawings, and from the claims.

The preceding is a simplified summary of the disclosure to provide an understanding of some aspects of the disclosure. This summary is neither an extensive nor exhaustive overview of the disclosure and its various aspects, implementations, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, implementations, and configurations of the disclosure are possible utilizing, alone or in combination, one or more of the features set forth above or described in detail below.

Numerous additional features and advantages of the present disclosure will become apparent to those skilled in the art upon consideration of the implementation descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, implementations, and configurations of the disclosure, as illustrated by the drawings referenced below.

FIG. 1 illustrates an example of a system according to at least one implementation of the present disclosure.

FIG. 2A illustrates an example according to at least one implementation of the present disclosure.

FIG. 2B illustrates an example according to at least one implementation of the present disclosure.

FIG. 3 illustrates an example of a process flow according to at least one implementation of the present disclosure.

### DETAILED DESCRIPTION

It should be understood that various aspects disclosed herein may be combined in different combinations than the combinations specifically presented in the description and accompanying drawings. It should also be understood that, depending on the example or implementation, certain acts or events of any of the processes or methods described herein may be performed in a different sequence, and/or may be added, merged, or left out altogether (e.g., all described acts or events may not be necessary to carry out the disclosed techniques according to different implementations of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any implementations of the disclosure are explained in detail, it is to be understood that the disclosure is not limited in its application to the details of construction and the arrangement of components set forth in the following description or illustrated in the drawings. The disclosure is capable of other implementations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

The vagus nerve, a cranial nerve, is the longest nerve of the autonomic nervous system in the human body and includes sensory and motor fibers. The vagus nerve is responsible for the regulation of internal organ functions (e.g., digestion, heart rate, respiratory rate, etc.), vasomotor activity, and certain reflex actions (e.g., coughing, sneezing, swallowing, and vomiting). Some medical treatment techniques include stimulating the vagus nerve (e.g., vagus nerve stimulation (VNS)) for treating or controlling a medical condition (e.g., epilepsy). In some VNS techniques, a cuff electrode is placed in the cervical vagus nerve of a subject (e.g., patient) and stimulated. In some cases, VNS may result in laryngopharyngeal symptoms or side effects such as hoarseness in voice and coughing. For example, the side effects may be due to resultant laryngeal muscle activation associated with VNS.

Some techniques for understanding and monitoring the side effects of VNS include asking the subject of his/her condition (e.g., sensory experience, perceptually) in response to applied stimulation signals. In some cases, electromyography techniques are applied for monitoring resultant muscle electromyographic (EMG) signals associated with VNS. For example, the EMG signals may be representative of a muscle response (e.g., laryngeal muscle contractions) to electrical stimulation pulses generated in association with VNS treatment. Muscle response may be referred to as muscle activity. The monitoring of EMG activity may support understanding side effects associated with VNS. In some cases, this can be through monitoring of immediately placed muscles (e.g., hyoid muscles, thyroid muscles, and laryngeal muscles relatively near the vagus nerve or branches thereof).

Aspects of the present disclosure support VNS using cuff electrodes in association with treating a medical condition (e.g., controlling epilepsy). According to example aspects of the present disclosure, a cuff electrode and associated side wing needles are described which support monitoring of EMG activity (e.g., monitoring of close by EMG) and understanding side effects of VNS based on the EMG activity. As described herein, the cuff electrode (also referred to herein as a nerve cuff, a stimulation and sensory cuff electrode, etc.) is provided for neural stimulation.

In an example aspect, a device includes cuff electrode suture wings with needle hooks supportive of monitoring EMG activity. In an example, a cuff electrode (or multiple cuff electrodes) is placed on a cervical vagus nerve and sutured to the nerve (e.g., using a suture wing). In some aspects, the needle hooks may stabilize placement of the cuff electrode (e.g., act as stabilizers). The needle hooks may be active electrodes capable of monitoring EMG activity from muscles located relatively close to a stimulated nerve (e.g., vagus nerve). For example, the needle hooks may support monitoring activity (e.g., EMG activity) of hyoid muscles, thyroid muscles, and laryngeal muscles. The needle hooks may be referred to as hook electrodes. An active electrode described herein includes integrated electronic circuitry (e.g., an integrated circuit chip or board) associated with measuring or monitoring activity (e.g., EMG activity, ENG activity, EKG activity, etc.). For example, the electronic circuitry may include a pre-amplifier circuit. In an example, an active electrode described herein may include a passive electrode and a pre-amplifier integrated within the same package or board.

The needle hooks (also referred to herein as hook electrodes and/or needle hook electrodes) may support the monitoring of nerve stimulation side effects (also referred to herein as biometric responses to the nerve stimulation). For example, a main intent of nerve stimulation is to stimulate and then activate a nerve (e.g., stimulate a nerve up and down). Aspects of the present disclosure support programming therapy stimulation for a subject based on monitoring and/or analyzing the side effects.

Implementations of the present disclosure provide technical solutions associated with monitoring side effects associated with nerve stimulation (e.g., VNS). For example, aspects of the present disclosure include a cuff electrode, needle hooks (e.g., hook electrodes), and a suture structure (e.g., a suture wing) to which both the cuff electrode and the needle hooks are coupled. Aspects of the present disclosure may support improved monitoring of side effects and benefits to programming therapy sessions for a subject.

In some other aspects, the needle hooks (e.g., hook electrodes) may provide increased stability associated with the suture structure, and thereby, the cuff electrode. For example, the needle hooks may support implementations that provide relatively stable (e.g., fixed) positioning of the cuff electrode, without affixing the suture structure to the tissue of a subject using a suturing material (e.g., sutures, thread, etc.). In an example, the needle hooks coupled to the suture structure may support implementations which refrain from using coupling elements (e.g., suturing material) for coupling or joining the suture structure to a tissue (e.g., muscle tissue) of the subject. Aspects described herein may support a cuff electrode implemented at a nerve, without an anchor component (e.g., a non-functioning extra cuff as a tethering anchor) at the same nerve (or nerve branch thereof). Accordingly, for example, aspects of the cuff electrode and needle hooks described herein may support decreased trauma to the nerve for instances in which the cuff electrode is replaced.

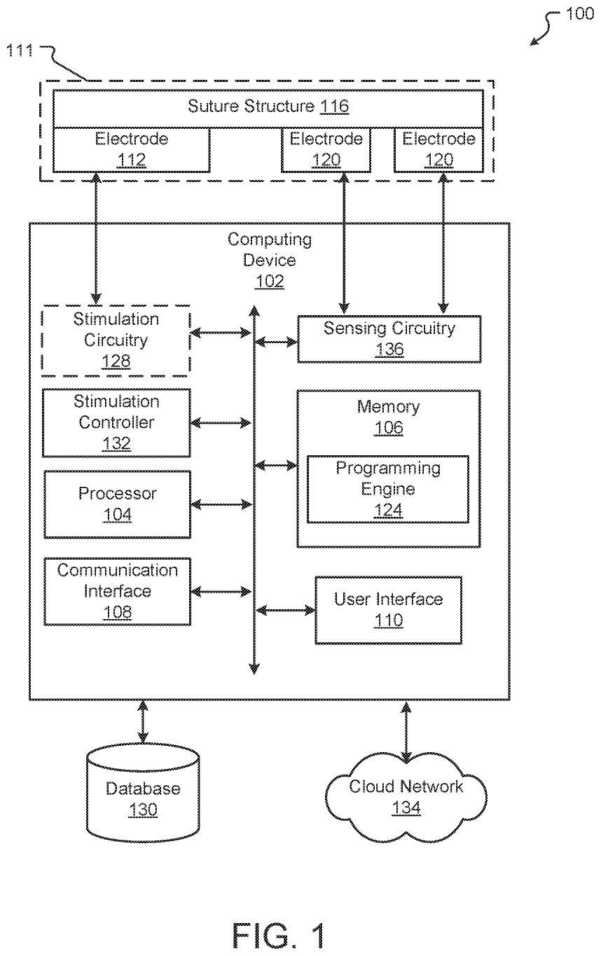

FIG. 1 illustrates an example of a system 100 that supports aspects of the present disclosure. The system 100 includes a computing device 102, an implantable device 111, a database 130, and/or a cloud network 134 (or other network). The implantable device 111 may include an electrode 112, suture structure 116, and electrode(s) 120. Systems according to other implementations of the present disclosure may include more or fewer components than the system 100. For example, the system 100 may omit and/or include additional instances of the implantable device 111 (e.g., the electrode 112, suture structure 116, electrode 120), one or more components of the computing device 102, the database 130, and/or the cloud network 134. The system 100 may support the implementation of one or more other aspects of one or more methods disclosed herein.

The computing device 102 includes a processor 104, a memory 106, a communication interface 108, and a user interface 110. Computing devices according to other implementations of the present disclosure may include more or fewer components than the computing device 102. The computing device 102 may be, for example, a control device including electronic circuitry (e.g., stimulation circuitry 128, stimulation controller 132) associated with driving the electrode 112. The computing device 102 may include electronic circuitry (e.g., sensing circuitry 136) associated with sensing signals output by the electrode(s) 120.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the electrode 112, the electrode 120, the database 130, and/or the cloud network 134.

The memory 106 may be or include RAM, DRAM, SDRAM, other solid-state memory, any memory described herein, or any other tangible, non-transitory memory for storing computer-readable data and/or instructions. The memory 106 may store information or data associated with completing, for example, any step of the process flow 300 described herein, or of any other methods. The memory 106 may store, for example, instructions and/or machine learning models that support one or more functions of the computing device 102. For instance, the memory 106 may store content (e.g., instructions and/or machine learning models) that, when executed by the processor 104, enable programming (e.g., by a programming engine 124) associated with nerve stimulation (e.g., neural stimulation). Such content, if provided as in instruction, may, in some implementations, be organized into one or more applications, modules, packages, layers, or engines.

Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the electrode 112, the electrode 120, the database 130, and/or the cloud network 134.

The computing device 102 may also include a communication interface 108. The communication interface 108 may be used for receiving data or other information from an external source (e.g., another computing device 102, the cloud network 134, and/or any other system or component separate from the system 100), and/or for transmitting instructions, data (e.g., measurements, temperature information, etc.), or other information to an external system or device (e.g., another computing device 102, the database 130, the cloud network 134, and/or any other system or component not part of the system 100). The communication interface 108 may include one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some implementations, the communication interface 108 may support communication between the device 102 and one or more other processors 104 or computing devices 102, whether to reduce the time needed to accomplish a computing-intensive task or for any other reason.

The computing device 102 may also include one or more user interfaces 110. The user interface 110 may be or include a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some implementations, the user interface 110 may support user modification (e.g., by a surgeon, medical personnel, a patient, etc.) of instructions to be executed by the processor 104 according to one or more implementations of the present disclosure, and/or to user modification or adjustment of a setting of other information displayed on the user interface 110 or corresponding thereto.

In some implementations, the computing device 102 may utilize a user interface 110 that is housed separately from one or more remaining components of the computing device 102. In some implementations, the user interface 110 may be located proximate one or more other components of the computing device 102, while in other implementations, the user interface 110 may be located remotely from one or more other components of the computer device 102.

The electrode 112 may be a cuff electrode. In some aspects, the electrode 112 may be a bipolar nerve cuff electrode. In some other aspects, the electrode 112 may be a monopolar nerve cuff electrode. The electrode 112 may be an electrode capable of delivering electrical stimulation signals (e.g., stimulation pulses) to a nerve (e.g., a vagus nerve, a nerve branch, a tibial nerve, other peripheral nerves, etc.) of the subject. In some aspects, the electrode 112 may be coupled to or integrated with an implanted stimulation lead (not illustrated) of the computing device 102.

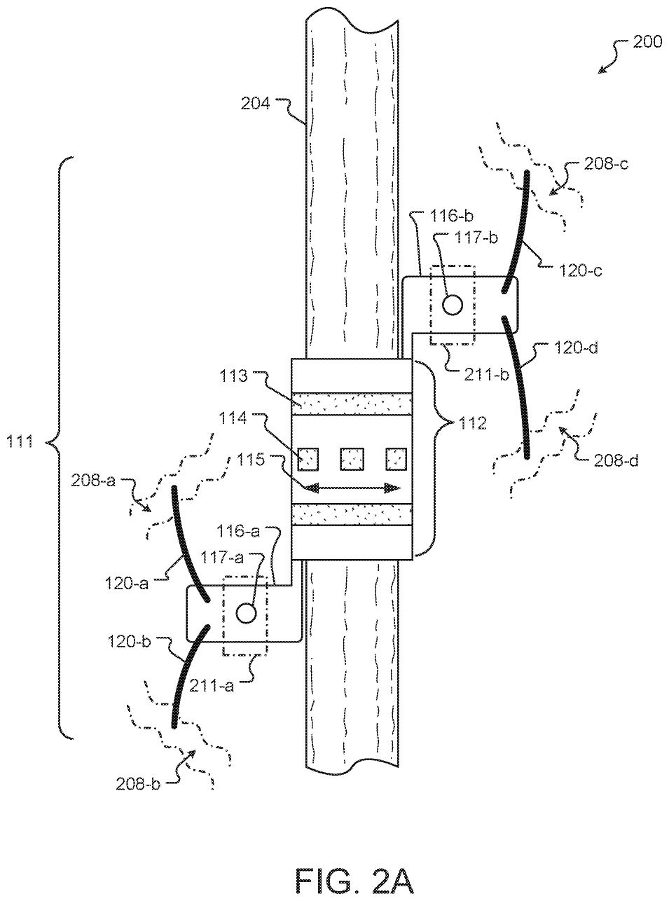

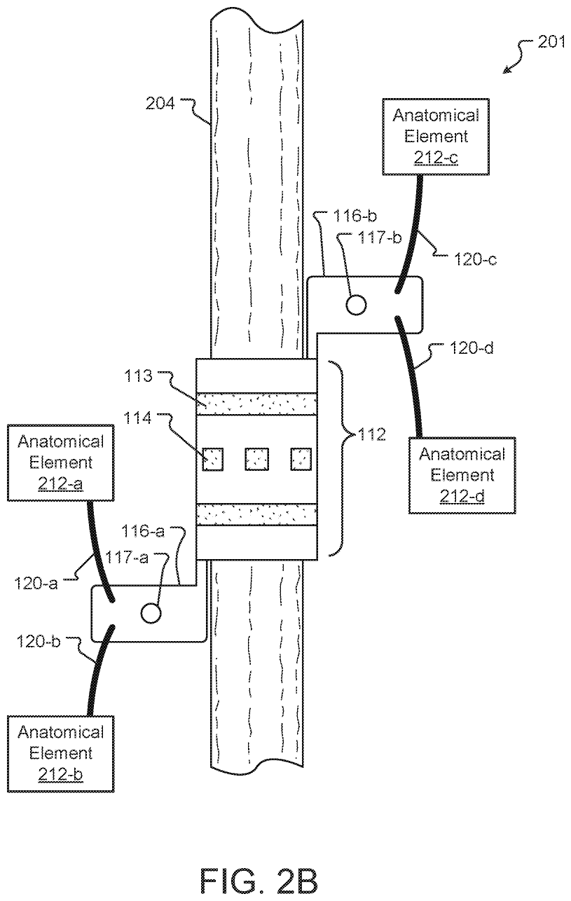

The suture structure 116 may be a suture wing. In some aspects, the suture structure 116 may be integrally formed with the electrode 112. Additionally, or alternatively, the suture structure 116 may be separate from the electrode 112 (e.g., separately formed) and attached to the electrode 112 with an attachment mechanism (e.g., friction fit, clasp, etc.). In an example, the attachment mechanism may be disposed at an intersection between a surface of the electrode 112 and the suture structure 116. Example aspects of the suture structure 116 are later described with reference to FIGS. 2A and 2B.

The electrode 120 may be a sensing electrode capable of detecting electrical activity produced by other anatomical elements (e.g., skeletal muscles, laryngeal muscles, the heart, nerves, etc.) of the subject. In some aspects, the electrode 120 may be a needle hook (e.g., needle electrode, hook electrode) as described herein. In some aspects, the electrode 120 may stabilize placement of the suture structure 116, and thereby, the electrode 112. That is, for example, the electrode 120 may act as a stabilizer. In some aspects, the electrode 112 and the electrode 120 may be physically coupled to the suture structure 116.

In an example, the electrode 120 may support sensing of stimulation evoked signals (e.g., signals responsive to electrical stimulation signals provided via the electrode 112). In an example, the electrode 120 may support sensing of EMG signals representative of a response of a muscle to the electrical stimulation signals. In another example, the electrode 120 may support sensing of electroneurographic (ENG) signals representative of a response of an anatomical element (e.g., muscles, the heart, nerves, etc.) to the electrical stimulation signals. In an example, the electrode 120 may support sensing of electrocardiogram (EKG) signals representative of a response heart to the electrical stimulation of a vagus nerve. In some aspects, the electrode 120 may be coupled to or integrated with an implanted sensing lead (not illustrated) of the computing device 102.

The stimulation circuitry 128 may be integrated with the computing device 102. Additionally, or alternatively, the stimulation circuitry 128 may be external to the computing device 102 and coupled (e.g., via a wired or wireless connection) to the computing device 102. The stimulation circuitry 128 may produce electrical stimulation signals (e.g., pulses) and deliver the electrical stimulation signals to the electrode 112.

The stimulation controller 132 may control delivery of the electrical stimulation signals to the subject. For example, the stimulation controller 132 may set and/or modify one or more stimulation parameters associated with delivering the stimulation signals. For example, the stimulation controller 132 may set and/or modify parameters such as duration (e.g., of a therapy session), frequency, duty cycle amplitude, pulse width, in association with the delivery of electrical stimulation signals.

The computing device 102 may be a medical device implanted in the body of a subject. For example, the computing device 102 may be a neurostimulation device (e.g., a neurostimulator) that includes the stimulation circuitry 128, the sensing circuitry 136, and the stimulation controller 132. In some examples, the computing device 102 may be an implanted neurostimulator (e.g., an implanted pulse generator (IPG))). In some cases, the computing device 102 may be a cardiac pacemaker, a cardioverter-defibrillator, a drug delivery device, a biologic therapy device, a monitoring or therapeutic device, etc. The computing device 102 may be integrated with or separate from any of the components (e.g., the stimulation circuitry 128, the sensing circuitry 136, and the stimulation controller 132, etc.) described with reference to the computing device 102.

The sensing circuitry 136 may be electrically coupled to electrode 120. The sensing circuitry 136 may sense signals detected by the electrode 120. In some aspects, the sensing circuitry 136 may determine or calculate parameters (e.g., frequency, amplitude, etc.) associated with the signals. For example, the sensing circuitry 136 may include circuitry configured to sense response signals representative of responses of the anatomical elements of the subject (e.g., stimulation-evoked responses).

In an example, the sensing circuitry 136 may include circuitry configured to sense response signals (e.g., ENG signals) representative of a response of anatomical elements (e.g., other nerves, etc.) due to electrical stimulation signals applied to the nerve via the electrode 112. In another example, the sensing circuitry 136 may include circuitry configured to sense signals (e.g., EMG signals) representative of a response of other anatomical elements (e.g., a muscle, an organ, etc.) to the electrical stimulation signals. In some aspects, the other anatomical elements may be innervated by a branch of the nerve stimulated by the electrical stimulation signals.

Example aspects of the electrode 120, the stimulation circuitry 128, the stimulation controller 132, and the sensing circuitry 136 are later described with reference to FIGS. 2A and 2B.

The programming engine 124 may calculate parameters described herein associated with the delivery of electrical stimulation signals. For example, the programming engine 124 may calculate parameters such as duration of a therapy session and parameters (e.g., frequency, duty cycle, amplitude, pulse width, etc.) of an applied stimulation signal.

The computing device 102 may support a closed-loop system associated with controlling nerve stimulation to treat various medical conditions described herein. For example, the stimulation controller 132 may be a closed-loop controller (also referred to herein as a closed-loop feedback controller) supportive of applying electrical stimulation to a nerve (or nerve branch) of the subject, and the computing device 102 may monitor a response of the subject to determine the efficacy of the stimulation. For example, the computing device 102 may monitor signals (e.g., EMG signals, ENG signals, EKG signals, blood glucose levels, etc.) representative of characteristics of a response of an anatomic element of the subject. Based on an analysis of the signals, the computing device 102 may determine an effectiveness of the delivered electrical stimulation (e.g., delivered therapy). In some aspects, using closed-loop feedback control, the computing device 102 may control or modulate the level of therapy delivered to the subject. That is, for example, the computing device 102 may provide therapy stimulation control for closed loop.

The database 130 may store information that correlates nerves stimulated by the computing device 102 and/or anatomical elements monitored in response to the stimulation. The database 130 may additionally or alternatively store, for example, location or coordinates of the computing device 102 and/or the implantable device 111. The database 130 may be configured to provide any such information to the computing device 102 or to any other device of the system 100 or external to the system 100, whether directly or via the cloud network 134. In some implementations, the database 130 may include information (e.g., a nerve stimulation plan) associated with diagnosing and/or treating a medical condition of a patient. In some implementations, the database 130 may be or comprise part of a hospital image storage system, such as a picture archiving and communication system (PACS), a health information system (HIS), and/or another system for collecting, storing, managing, and/or transmitting electronic medical records including image data.

In some aspects, the computing device 102 may communicate with a server(s) and/or a database (e.g., database 130) directly or indirectly over a communications network (e.g., the cloud network 134). The communications network may include any type of known communication medium or collection of communication media and may use any type of protocols to transport data between endpoints. The communications network may include wired communications technologies, wireless communications technologies, or any combination thereof.

Wired communications technologies may include, for example, Ethernet-based wired local area network (LAN) connections using physical transmission mediums (e.g., coaxial cable, copper cable/wire, fiber-optic cable, etc.). Wireless communications technologies may include, for example, cellular or cellular data connections and protocols (e.g., digital cellular, personal communications service (PCS), cellular digital packet data (CDPD), general packet radio service (GPRS), enhanced data rates for global system for mobile communications (GSM) evolution (EDGE), code division multiple access (CDMA), single-carrier radio transmission technology (1×RTT), evolution-data optimized (EVDO), high speed packet access (HSPA), universal mobile telecommunications service (UMTS), 3G, long term evolution (LTE), 4G, and/or 5G, etc.), Bluetooth®, Bluetooth® low energy, Wi-Fi, radio, satellite, infrared connections, and/or ZigBee® communication protocols.

The Internet is an example of the communications network that constitutes an Internet Protocol (IP) network consisting of multiple computers, computing networks, and other communication devices located in multiple locations, and components in the communications network (e.g., computers, computing networks, communication devices) may be connected through one or more telephone systems and other means. Other examples of the communications network may include, without limitation, a standard Plain Old Telephone System (POTS), an Integrated Services Digital Network (ISDN), the Public Switched Telephone Network (PSTN), a Local Area Network (LAN), a Wide Area Network (WAN), a wireless LAN (WLAN), a Session Initiation Protocol (SIP) network, a Voice over Internet Protocol (VoIP) network, a cellular network, and any other type of packet-switched or circuit-switched network known in the art. In some cases, the communications network 120 may include of any combination of networks or network types. In some aspects, the communications network may include any combination of communication mediums such as coaxial cable, copper cable/wire, fiber-optic cable, or antennas for communicating data (e.g., transmitting/receiving data).

The computing device 102 may be connected to the cloud network 134 via the communication interface 108, using a wired connection, a wireless connection, or both. In some implementations, the computing device 102 may communicate with the database 130 and/or an external device (e.g., a computing device) via the cloud network 134.

The system 100 or similar systems may be used, for example, to carry out one or more aspects of any of the methods (e.g., process flow 300) described herein. The system 100 or similar systems may also be used for other purposes.

FIG. 2A illustrates an example 200 of the system 100 (e.g., implantable device 111) described herein. For example, FIG. 2A illustrates aspects of electrode 112, suture structure 116, electrode 120 described herein.

According to example aspects of the present disclosure, the electrode 112 is a cuff electrode clamped to a nerve 204 of a subject. The nerve 204 may be, for example, a vagus nerve or a nerve branch thereof. For example, the nerve 204 may be a cervical vagus nerve (or nerve branch thereof) of the subject. Aspects of the present disclosure described herein may be applied to nerves (and nerve branches thereof) other than the vagus nerve. For example, aspects of the present disclosure may be applied to stimulating any peripheral nerves (e.g., a tibial nerve, etc.) of the body of the subject and monitoring responses from surrounding tissues (e.g., tissue 208 later described herein). The electrode 112 may be coupled to one or more suture structures 116. For example, in the example illustrated in FIG. 2A, the electrode 112 is coupled to suture structure 116-a and suture structure 116-b.

The electrode 112 may be, for example, a tubular structure or semi-tubular structure formed of an autoclavable material (e.g., silicone rubber, Teflon, stainless steel, etc.). The electrode 112 may include one or more electrode contacts 113 (also referred to herein as circumferential cuff electrode contacts) and one or more electrical contacts 114 (also referred to herein as partially circumferential electrode contacts). One or more dimensions (e.g., in a circumferential direction 115) of the electrode contacts 113 may, for example, correspond to a circumference of the electrode 112. One or more dimensions (e.g., in the circumferential direction 115) of the electrode contacts 114 may, for example, be less than the circumference of the electrode 112. The electrode contacts 113 and electrode contacts 114 may support recording or providing stimulation at locations or points along the nerve 204.

Each suture structure 116 (e.g., suture structure 116-a, suture structure 116-b) may have one or more electrodes 120 coupled thereto. The electrodes 120 may be, for example, needle electrodes (e.g., needle hooks) as described herein. In an example, electrode 120-a and electrode 120-b are coupled to suture structure 116-a. In some cases, electrode 112-a and electrode 112-b may be located at respective wings (also referred to herein as sides) of the suture structure 116-a. Additionally, or alternatively, electrode 112-a and electrode 112-b may be located at the same wing (or side) of the suture structure 116-a. In another example, electrode 120-c and electrode 120-d are coupled to suture structure 116-b. Electrode 112-c and electrode 112-d may be located at respective wings of the suture structure 116-b or at the same wing of the suture structure 116-b.

In some aspects, each suture structure 116 (e.g., suture structure 116-a, suture structure 116-b) has one or more openings 117 via which the suture structure 116 may be attached to a subject. For example, each suture structure 116 may be attached to tissue 211 (e.g., muscle tissue, nerve tissue, etc.) of the subject using coupling elements (not illustrated) such as suturing material (e.g., thread, fiber, etc.). In an example, the suture structure 116-a may be attached to the tissue 211-a using a coupling element (e.g., a suturing material) and/or the suture structure 116-b may be attached to the tissue 211-b using a coupling element. In some cases, tissue 211-a and/or tissue 211-b may be tissue (e.g., tissue 208) of an anatomical element 212 later described herein.

Additionally, or alternatively, the suture structures 116 may support implementations which refrain from using coupling elements (e.g., suturing material) for coupling or joining the suture structures 116 to the subject. In an example implementation, the electrode 120-a through electrode 120-d provide substantial stability with respect to a physical placement and/or positioning of the suture structures 116-a, the suture structure 116-b, and correspondingly, the electrode 112. Accordingly, for example, the suture structure 116-a and the suture structure 116-b may be implemented without coupling elements (e.g., suturing material) at opening 117-a and opening 117-b, respectively.

The present disclosure supports alternative and/or additional implementations in which, for an electrode 112 coupled to a combination of suture structures 116, one or more of the suture structures 116 (e.g., suture structure 116-a) is implemented with coupling elements at a corresponding opening 117 (e.g., opening 117-a), and others of the suture structures 116 (e.g., suture structure 116-b) are implemented without coupling elements at a corresponding opening 117 (e.g., opening 117-b).

Tissue 208 (e.g., tissue 208-a through tissue 208-d) may be, for example, muscle tissue of an anatomical element 212 (later illustrated in FIG. 2B) of the subject. In some aspects, the tissue 208 may be tissue associated with a nerve of an anatomical element 212. In an example, tissue 208-a and tissue 208-b may include be surrounding the suture structure 116-a, and tissue 208-c and tissue 208-d may be tissue surrounding the suture structure 116-b. Example aspects of the tissue 208 are described later herein with respect to FIG. 2B.

FIG. 2B illustrates an example 201 of the system 100 described herein.

According to example aspects of the present disclosure, the computing device 102 (e.g., stimulation circuitry 128) may deliver electrical stimulation signals to the nerve 204 (e.g., via the electrode 112). Additionally, or alternatively, the computing device 102 may deliver electrical stimulation signals to one or more branches (not illustrated) of the nerve 204.

The computing device 102 (e.g., sensing circuitry 136) may sense signals associated with an anatomical element 212 (e.g., anatomical element 212-a, anatomical element 212-b, anatomical element 212-c, anatomical element 212-d, etc.). The anatomical element 212 may be any anatomical element of the subject. For example, the anatomical element 212 may be a nerve, a muscle, an organ, or the like, and is not limited thereto.

The computing device 102 may detect the signals using an electrode 120 coupled to the anatomical element 212 and/or tissue 208 (not illustrated for simplicity) of the anatomical element 212. In some aspects, the electrode 120 may be coupled a nerve of the anatomical element 212. The sensed signals may be representative of a response (e.g., a muscle response, muscle activity, etc.) of an anatomical element 212 that corresponds to the stimulation signals (e.g., pulses) provided to the nerve 204.

The sensing circuitry 136 may record a signal representative of characteristics of the response of the anatomical element 212. For example, parameter values of the signal (e.g., EMG, ENG, EKG, etc.) may represent characteristics of the response to the stimulus delivered by 112.

The programming engine 124 may analyze the parameter values (e.g., EMG, EKG, ENG, etc.) of the signal from the anatomical element 212. Based on the analysis, the programming engine 124 may modify stimulus parameters (e.g., amplitude, pulse width, frequency etc.) based on the analysis. For example, the programming engine 124 may modify and/or confirm (e.g., maintain) the stimulus parameters in association with treating a neuromodulation treatable disease condition. In some examples, the programming engine 124 may modify and/or confirm the stimulus parameters in association with treating different neuromodulation treatable disease conditions such as epilepsy. In some examples, the programming engine 124 may modify the stimulus parameters based on response signals from anatomical element 212-a through anatomical element 212-d (e.g., as recorded by the sensing circuitry 136).

In some aspects, based on the analysis, the programming engine 124 may set and/or adjust parameters associated with delivering the electrical stimulation signals (e.g., amplitude, pulse width, frequency etc.) to the nerve 204. In some examples, the programming engine 124 may adjust one or more of the parameters (e.g., amplitude, pulse width, frequency etc.) based on a comparison of the characteristics (e.g., of the signal generated by the sensing circuitry 136) to a set of criteria associated with treating or managing a medical condition of the subject. In an example, treating or managing a medical condition may include controlling seizure in epileptic patients, a cardiovascular function, controlling blood glucose levels in association with diabetes management, or the like. Example criteria may include, and are not limited to, an amplitude threshold value, a frequency threshold value, a pulse width threshold value, amplitude with respect to a temporal period, frequency with respect to a temporal period, or the like.

In some other aspects, based on the analysis, the programming engine 124 may set and/or adjust a duration of a therapy session associated with treating the medical condition. In another example, based on the analysis, the programming engine 124 may calculate set and/or adjust parameters (e.g., frequency, duty cycle, amplitude, pulse width, etc.) of an applied electrical stimulation signal. In some cases, the programming engine 124 may provide notifications (e.g., via user interface 110) to a subject (e.g., patient), healthcare personnel, or the like regarding the programming.

In an example implementation, for electrical stimulation signals delivered to the nerve 204 via the electrode 112, the computing device 102 (e.g., using electrode 120-a and sensing circuitry 136) may sense an ENG signal indicative of a corresponding response of the anatomical element 212-a. For example, the anatomical element 212-a may be a nerve trunk of the nerve 204.

In another example implementation, for electrical stimulation signals delivered to the nerve 204 via the electrode 112, the computing device 102 (e.g., using electrode 120-b and sensing circuitry 136) may sense an EMG signal indicative of a corresponding response of the anatomical element 212-b. For example, the anatomical element 212-b may be a laryngeal muscle, a hyoid muscle, or a thyroid muscle of the subject.

In another example implementation, for electrical stimulation signals delivered to the nerve 204 via the electrode 112, the computing device 102 (e.g., using electrode 120-c and sensing circuitry 136) may sense an EKG signal indicative of a corresponding response of the anatomical element 212-c. For example, the anatomical element 212-c may be a thoracic organ (e.g., the heart) of the subject. In some aspects, based on the EKG signal, the computing device 102 (e.g., programming engine 124) may calculate or determine biometric information of the subject. For example, based on the EKG signal, the computing device 102 may calculate or determine blood glucose information (e.g., blood glucose levels) of the subject.

In some other example implementations, for electrical stimulation signals delivered to the nerve 204 via the electrode 112, the computing device 102 (e.g., using electrode 120-d and sensing circuitry 136) may sense blood glucose levels associated with the subject.

In another example implementation, for electrical stimulations signals delivered to the nerve 204 via the electrode 112, the computing device 102 (e.g., using electrode 120-c and sensing circuitry 136) may sense blood glucose level indicative of a corresponding response anatomical structure (nerve) to the stimulating signals.

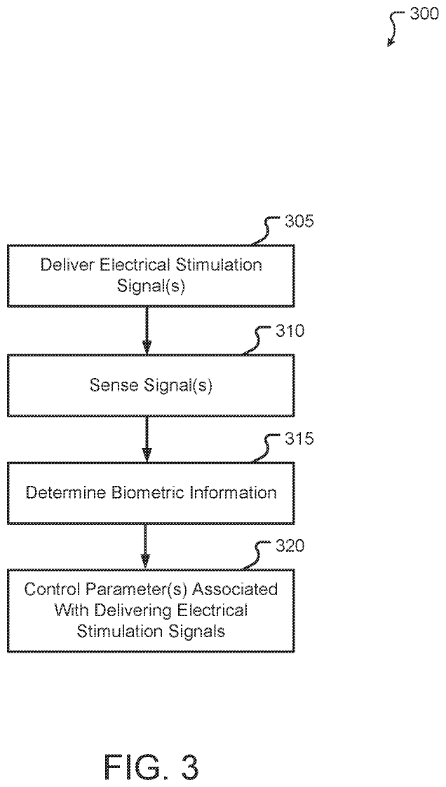

FIG. 3 illustrates an example of a process flow 300 in accordance with aspects of the present disclosure. In some examples, process flow 300 may be implemented by aspects of the system 100. For example, process flow 300 may be implemented by aspects of a computing device 102, an implantable device 111 (e.g., electrode 112, suture structure 116, electrode 120), stimulation circuitry 128, stimulation controller 132, sensing circuitry 136, etc.) described with reference to FIGS. 1 through 3.

In the following description of the process flow 300, the operations may be performed in a different order than the order shown, or the operations may be performed in different orders or at different times. Certain operations may also be left out of the process flow 300, or other operations may be added to the process flow 300.

It is to be understood that any of the operations of process flow 300 may be performed by any device (e.g., a computing device 102, components thereof, etc.).

At 305, the process flow 300 may include delivering, via a first electrode (e.g., electrode 112), one or more electrical stimulation signals to a nerve (e.g., nerve 204) or a nerve branch of the nerve. In some aspects, the first electrode includes a cuff electrode. In an example, the nerve and nerve branches are associated with a subject.

At 310, the process flow 300 may include sensing, via one or more second electrodes (e.g., electrodes 120), one or more signals based on delivering the one or more electrical stimulation signals. In some aspects, the first electrode and the one or more second electrodes are both coupled to a suture structure (e.g., suture structure 116).

In some aspects, the one or more second electrodes include one or more needle electrodes. In some aspects, the one or more second electrodes are coupled to tissue (e.g., tissue 208) of the subject.

In some aspects, the suture structure includes a suture wing structure.

In an example, the one or more signals include one or more EMG signals.

In another example, the one or more signals include one or more ENG signals.

In some examples, the one or more signals include one or more EKG signals.

In some aspects, the one or more signals are representative of a feedback response of an anatomical element of a subject corresponding to the one or more electrical stimulation signals.

At 315, the process flow 300 may include determining biometric information associated with the subject based at least in part on the one or more signals.

At 320, the process flow 300 may include controlling one or more parameters associated with delivering the one or more electrical stimulation signals, based on sensing (e.g., at 310) the one or more signals. In some aspects, controlling the one or more parameters may be based on determining (e.g., at 315) the biometric information.

In an example, the one or more parameters include at least one of: a duration associated with delivering the one or more electrical stimulation signals; a frequency associated with delivering the one or more electrical stimulation signals; a duty cycle associated with delivering the one or more electrical stimulation signals; and an amplitude associated with delivering the one or more electrical stimulation signals.

The process flow 300 (and/or one or more operations thereof) may be carried out or otherwise performed, for example, by at least one processor. The at least one processor may be the same as or similar to the processor(s) 104 of the computing device 102 described above. A processor other than any processor described herein may also be used to execute the process flow 300. The at least one processor may perform operations of the process flow 300 by executing elements stored in a memory such as the memory 106. The elements stored in memory and executed by the processor may cause the processor to execute one or more operations of a function as shown in the process flow 300. One or more portions of the process flow 300 may be performed by the processor executing any of the contents of memory, such as a programming engine 124.

As noted above, the present disclosure encompasses methods with fewer than all of the steps identified in FIG. 3 (and the corresponding description of the process flow 300), as well as methods that include additional steps beyond those identified in FIG. 3 (and the corresponding description of the process flow 300). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, implementations, and/or configurations for the purpose of streamlining the disclosure. The features of the aspects, implementations, and/or configurations of the disclosure may be combined in alternate aspects, implementations, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, implementation, and/or configuration. Thus, the following claims are hereby incorporated into this Detailed Description, with each claim standing on its own as a separate preferred implementation of the disclosure.

Moreover, though the foregoing has included description of one or more aspects, implementations, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, implementations, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

Example aspects of the present disclosure include:

A device including: a cuff electrode; one or more needle electrodes; and a suture structure, where the cuff electrode and the one or more needle electrodes are both coupled to the suture structure.

Any of the aspects herein, wherein the suture structure includes a suture wing structure.

Any of the aspects herein, wherein the one or more needle electrodes include one or more needle hook electrodes; and a stability associated with a placement of the device is associated with the needle hook electrodes.

Any of the aspects herein, wherein the one or more needle electrodes include one or more active needle electrodes.

Any of the aspects herein, further including: electronic circuitry configured to: deliver, via the first electrode, one or more electrical stimulation signals to a nerve or a nerve branch; sense, via the one or more second electrodes, one or more signals based on delivering the one or more electrical stimulation signals; and control one or more parameters associated with delivering the one or more electrical stimulation signals, based on sensing the one or more signals.

Any of the aspects herein, wherein the one or more signals include one or more EMG signals.

Any of the aspects herein, wherein the one or more signals include one or more ENG signals.

Any of the aspects herein, wherein the one or more signals include one or more EKG signals.

Any of the aspects herein, wherein the one or more signals are representative of a blood glucose level.

A system including: a first electrode; one or more second electrodes; a suture structure, where the first electrode and the one or more second electrodes are both coupled to the suture structure; a processor; and a memory storing data thereon that, when processed by the processor, cause the processor to: deliver, via the first electrode, one or more electrical stimulation signals to a nerve; sense, via the one or more second electrodes, one or more signals based on delivering the one or more electrical stimulation signals; and control one or more parameters associated with delivering the one or more electrical stimulation signals, based on sensing the one or more signals.

Any of the aspects herein, wherein the suture structure includes a suture wing structure.

Any of the aspects herein, wherein the first electrode includes a cuff electrode.

Any of the aspects herein, wherein the one or more signals include one or more EMG signals.

Any of the aspects herein, wherein the one or more signals include one or more ENG signals.

Any of the aspects herein, wherein the one or more signals include one or more EKG signals.

Any of the aspects herein, wherein the one or more signals are representative of a blood glucose level.

Any of the aspects herein, wherein the one or more signals are representative of a feedback response of an anatomical element of a subject corresponding to the one or more electrical stimulation signals.

Any of the aspects herein, wherein the data, when processed by the processor, further causes the processor to: determine biometric information associated with a subject based on the one or more signals, where controlling the one or more parameters is based on determining the biometric information.

Any of the aspects herein, wherein the biometric information includes one or more biometric responses of the subject corresponding to the one or more electrical stimulation signals, the one or more biometric responses including at least one of: a neural response of the subject; a change to a heart rate of the subject; and one or more laryngopharyngeal symptoms of the subject.

Any of the aspects herein, wherein delivering the one or more electrical stimulation signals, sensing the one or more signals, and controlling the one or more parameters associated with delivering the one or more electrical stimulation signals is based on closed-loop control.

Any of the aspects herein, wherein the data, when processed by the processor, further causes the processor to: generate status information associated with a subject based on sensing the one or more signals, controlling the one or more parameters, or both; and provide at least a portion of the clinical data to the subject, a medical provider, or both.

Any of the aspects herein, wherein the one or more second electrodes include one or more needle electrodes.

Any of the aspects herein, wherein: the one or more second electrodes include one or more needle hook electrodes; and a stability associated with a placement of the first electrode and the suture structure is associated with the needle hook electrodes.

Any of the aspects herein, wherein the one or more second electrodes include one or more active electrodes.

Any of the aspects herein, wherein controlling the one or more parameters includes modifying at least one of: a duration associated with delivering the one or more electrical stimulation signals; a frequency of the one or more electrical stimulation signals; a pulse width of the one or more electrical stimulation signals; a duty cycle of the one or more electrical stimulation signals; and an amplitude of the one or more electrical stimulation signals.

A method including: delivering, via a first electrode, one or more electrical stimulation signals to a nerve; sensing, via one or more second electrodes, one or more signals based on delivering the one or more electrical stimulation signals, where the first electrode and the one or more second electrodes are both coupled to a suture structure; and controlling one or more parameters associated with delivering the one or more electrical stimulation signals, based on sensing the one or more signals.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an implementation that is entirely hardware, an implementation that is entirely software (including firmware, resident software, micro-code, etc.) or an implementation combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. A device comprising:
a cuff electrode;
one or more needle electrodes; and
a suture structure, wherein the cuff electrode and the one or more needle electrodes are both coupled to the suture structure.

2. The device of claim 1, wherein the suture structure comprises a suture wing structure.

3. The device of claim 1, wherein:
the one or more needle electrodes comprise one or more needle hook electrodes; and
a stability associated with a placement of the device is associated with the needle hook electrodes.

4. The device of claim 1, wherein:
the one or more needle electrodes comprise one or more active needle electrodes.

5. The device of claim 1, further comprising:
electronic circuitry configured to:
deliver, via the first electrode, one or more electrical stimulation signals to a nerve or a nerve branch;
sense, via the one or more second electrodes, one or more signals based at least in part on delivering the one or more electrical stimulation signals; and
control one or more parameters associated with delivering the one or more electrical stimulation signals, based at least in part on sensing the one or more signals.

6. The device of claim 5, wherein the one or more signals comprise at least one of:
one or more electromyographic (EMG) signals;
one or more electroneurographic (ENG) signals; and
one or more electrocardiogram (EKG) signals.

7. The device of claim 5, wherein the one or more signals are representative of a blood glucose level.

8. A system comprising:
a first electrode;
one or more second electrodes;
a suture structure, wherein the first electrode and the one or more second electrodes are both coupled to the suture structure;
a processor; and
a memory storing data thereon that, when processed by the processor, cause the processor to:
deliver, via the first electrode, one or more electrical stimulation signals to a nerve;
sense, via the one or more second electrodes, one or more signals based at least in part on delivering the one or more electrical stimulation signals; and
control one or more parameters associated with delivering the one or more electrical stimulation signals, based at least in part on sensing the one or more signals.

9. The system of claim 8, wherein the suture structure comprises a suture wing structure.

10. The system of claim 8, wherein the first electrode comprises a cuff electrode.

11. The system of claim 8, wherein the one or more signals comprise at least one of:
one or more electromyographic (EMG) signals;
one or more electroneurographic (ENG) signals; and
one or more electrocardiogram (EKG) signals.

12. The system of claim 8, wherein the one or more signals are representative of at least one of:
a blood glucose level; and
a feedback response of an anatomical element of a subject corresponding to the one or more electrical stimulation signals.

13. The system of claim 8, wherein the data, when processed by the processor, further causes the processor to:
determine biometric information associated with a subject based at least in part on the one or more signals,
wherein controlling the one or more parameters is based at least in part on determining the biometric information.

14. The system of claim 13, wherein the biometric information comprises one or more biometric responses of the subject corresponding to the one or more electrical stimulation signals, the one or more biometric responses comprising at least one of:
a neural response of the subject;
a change to a heart rate of the subject; and
one or more laryngopharyngeal symptoms of the subject.

15. The system of claim 8, wherein delivering the one or more electrical stimulation signals, sensing the one or more signals, and controlling the one or more parameters associated with delivering the one or more electrical stimulation signals is based at least in part on closed-loop control.

16. The system of claim 8, wherein the data, when processed by the processor, further causes the processor to:
generate status information associated with a subject based at least in part on sensing the one or more signals, controlling the one or more parameters, or both; and
provide at least a portion of the clinical data to the subject, a medical provider, or both.

17. The system of claim 8, wherein the one or more second electrodes comprise at least one of:
one or more needle electrodes; and
one or more active electrodes.

18. The system of claim 8, wherein:
the one or more second electrodes comprise one or more needle hook electrodes; and
a stability associated with a placement of the first electrode and the suture structure is associated with the needle hook electrodes.

19. The system of claim 8, wherein controlling the one or more parameters comprises modifying at least one of:
a duration associated with delivering the one or more electrical stimulation signals;
a frequency of the one or more electrical stimulation signals;
a pulse width of the one or more electrical stimulation signals;
a duty cycle of the one or more electrical stimulation signals; and
an amplitude of the one or more electrical stimulation signals.

20. A method comprising:
delivering, via a first electrode, one or more electrical stimulation signals to a nerve;
sensing, via one or more second electrodes, one or more signals based at least in part on delivering the one or more electrical stimulation signals, wherein the first electrode and the one or more second electrodes are both coupled to a suture structure; and
controlling one or more parameters associated with delivering the one or more electrical stimulation signals, based at least in part on sensing the one or more signals.
